(19) United States
(12) Patent Application Publication
(10) Pub. No.: US 2013/0251793 A1
(43) Pub. Date: Sep. 26, 2013

(54) PHARMACEUTICAL COMPOSITION COMPRISING PHENTERMINE AND TOPIRAMATE

(71) Applicant: RANBAXY LABORATORIES LIMITED, New Delhi (IN)

(72) Inventors: Anuj Kumar FANDA, Ghaziabad (IN); Kumaravel VIVEK, Chennai (IN); Ravish SHARMA, Khargone (IN); Romi Barat SINGH, Varanasi (IN); Ajay Kumar SINGLA, Gurgaon (IN)

(73) Assignee: RANBAXY LABORATORIES LIMITED, New Delhi (IN)

(21) Appl. No.: 13/848,476

(22) Filed: Mar. 21, 2013

(30) Foreign Application Priority Data
Mar. 21, 2012 (IN) 826/DEL/2012

(51) Int. Cl. A61K 9/48; A61K 31/35; A61K 31/137

(52) U.S. Cl. CPC A61K 9/4808; A61K 31/137; A61K 31/35
USPC 424/457; 514/455; 424/400; 424/472

(57) ABSTRACT

This invention relates to a pharmaceutical composition comprising a combination of phentermine in an immediate-release form and topiramate in an extended-release form. Further, it relates to processes for the preparation of the composition and a method of using the composition.

### FIELD OF THE INVENTION

This invention relates to a pharmaceutical composition comprising a combination of phentermine in an immediate-release form and topiramate in an extended-release form. Further, it relates to processes for the preparation of the composition and a method of using the composition.

### BACKGROUND OF THE INVENTION

The prevalence of obesity in both children and adults is on the rise in first world countries, especially in the United States, as well as in many developing countries such as China and India. Many aspects of a person's life are affected by obesity, leading to physical problems such as knee and ankle joint deterioration. The medical problems caused by obesity can be serious and often life-threatening and include diabetes, shortness of breath and other respiratory problems such as asthma and pulmonary hypertension, gall bladder disease, dyslipidemia (for example, high cholesterol or high levels of triglycerides), dyslipidemic hypertension, osteoarthritis and other orthopedic problems, reflux esophagitis (heartburn), snoring, sleep apnea, menstrual irregularities, infertility, problems associated with pregnancy, gout, cardiovascular problems such as coronary artery disease and other heart trouble, muscular dystrophy, and metabolic disorders such as hypoalphalipoproteinemia, familial combined hyperlipidemia, and syndrome X, including insulin-resistant syndrome X. In addition, obesity has been associated with an increased incidence of certain cancers, notably cancers of the colon, rectum, prostate, breast, uterus, and cervix.

Strategies for treating obesity and related disorders have included dietary restriction, increased physical activity, pharmacological approaches, and even surgery, with the choice depending, at least in part, on the degree of weight loss one is attempting to achieve as well as on the severity of obesity exhibited by the subject.

Phentermine has been used as monotherapy in the treatment of obesity for about 30 years. Phentermine acts on the hypothalamus, an appetite control center of the brain. Phentermine monotherapy can increase weight loss when used in combination with diet and exercise, as compared to diet and exercise alone. However, the drug loses effectiveness after about two weeks and is not approved by the FDA for use beyond six weeks. Moreover, weight loss may not be permanent, especially after the drug is discontinued. Phentermine treatment is also associated with side effects including nervousness, irritability, headache, sweating, dry-mouth, nausea, and constipation.

The above-listed shortcomings encouraged the use of phentermine in combination with other agents. Various combination therapies that include phentermine as one of the agents have been investigated and have met with mixed success. More recently, it has been suggested that phentermine in combination with an anti-convulsant is a potentially effective therapy for effecting weight loss.

Combination therapy for effecting weight loss and treating obesity with a sympathomimetic agent (e.g., phentermine or a phentermine-like drug) and an anticonvulsant sulfamate derivative (e.g., topiramate) is disclosed in U.S. Pat. Nos. 7,674,776, 7,659,256, 7,553,818, and 7,056,890. A disclosed preferred embodiment of pharmaceutical compositions in these patents includes phentermine in an immediate-release formulation, and further includes topiramate in a controlled-release formulation.

U.S. Publication No. 2008/0113026 discloses a layered pharmaceutical formulation including two or more pharmaceutical layers and an intermediate layer disposed between the two or more pharmaceutical layers. A formulation wherein the first pharmaceutical layer comprises phentermine and the second pharmaceutical layer comprises topiramate is disclosed.

U.S. Publication No. 2008/0085306 discloses a delayed/extended-release formulation of topiramate for once-a-day administration.

U.S. Publication Nos. 2009/0304789 and 2009/0304785 disclose a controlled-release composition for treating obesity, diabetes, or a related condition in a subject comprising topiramate, microcrystalline cellulose, and methylcellulose. The publications also disclose a method for treating obesity and/or effecting weight loss by administering topiramate and optionally, in addition, a sympathomimetic agent such as phentermine.

U.S. Publication No. 2006/0121112 discloses a topiramate formulation with an immediate-release component of topiramate and a delayed-release component of topiramate.

U.S. Publication No. 2008/0118557 discloses a sustained-release topiramate formulation comprising a topiramate-containing enhanced immediate-release (EIR) component and an extended-release (XR) component.

U.S. Publication No. 2012/0003312 discloses multilayer minitablets comprising an immediate-release layer of phentermine and a modified-release layer of topiramate.

There is a need in the art to develop drug formulations in which phentermine is present in an immediate-release form and topiramate is present in an extended-release form that involve simple methods of production and are cost effective.

### SUMMARY OF THE INVENTION

The object of the present invention is to provide a pharmaceutical composition comprising phentermine in an immediate-release (IR) form and topiramate in an extended-release (ER) form.

Accordingly in one aspect, the present invention relates to a pharmaceutical composition of phentermine and topiramate comprising:
(i) an extended-release portion comprising a therapeutically effective amount of topiramate or its pharmaceutically acceptable salt, one or more rate-controlling agents, and one or more pharmaceutically acceptable excipients;
(ii) an immediate-release portion comprising a therapeutically effective amount of phentermine or its pharmaceutically acceptable salt and one or more pharmaceutically acceptable excipients.

Embodiments of the composition may include one or more of the following features. For example, the extended-release topiramate portion and the immediate-release phentermine portion may be present as cores. The cores may be in the form of pellets, granules, beads, minitablets, or tablets.

In one embodiment, the extended-release topiramate portion is present as cores and the immediate-release phentermine portion is present as a coating over the extended-release portion.

In another embodiment, the extended-release topiramate cores are present as matrix cores with an optional extended-release polymer coating over the cores.

In another embodiment, the extended-release topiramate cores are present as reservoir cores produced by coating the immediate-release topiramate cores with an extended-release polymer coating.

In another embodiment, the pharmaceutical composition of the present invention may be a hard gelatin capsule or a tablet.

In another aspect, the present invention relates to a process of preparing a pharmaceutical composition of phentermine and topiramate, wherein the process comprises the steps of:
(i) preparing extended-release topiramate cores comprising a therapeutically effective amount of topiramate or its pharmaceutically acceptable salt, one or more rate-controlling agents, and one or more pharmaceutically acceptable excipients;
(ii) preparing immediate-release phentermine cores comprising a therapeutically effective amount of phentermine or its pharmaceutically acceptable salt and one or more pharmaceutically acceptable excipients; and
(iii) combining the extended-release topiramate cores and the immediate-release phentermine cores.

In one embodiment, the extended-release topiramate cores are prepared by processes known in the art, such as compaction, dry granulation, wet granulation, fluidized bed granulation, and extrusion-spheronization.

In another embodiment the extended-release topiramate cores are prepared by coating the immediate-release topiramate cores with a layer of one or more rate-controlling agents.

In another embodiment, the immediate-release topiramate cores are prepared by extrusion-spheronization.

In another embodiment, the immediate-release topiramate cores are prepared by layering topiramate solution/dispersion over inert cores.

In another embodiment, the extended-release topiramate cores and the immediate-release phentermine cores are blended and encapsulated into a hard gelatin capsule.

In another embodiment, the extended-release topiramate cores and the immediate-release phentermine cores are blended and compressed into a tablet. The tablet may be a monolithic tablet, bilayered tablet, inlay tablet, or a compression coated tablet.

In another aspect, the present invention relates to a process of preparing a pharmaceutical composition of phentermine and topiramate, wherein the process comprises the steps of:
(i) preparing extended-release topiramate cores comprising a therapeutically effective amount of topiramate or its pharmaceutically acceptable salt, one or more rate-controlling agents, and one or more pharmaceutically acceptable excipients;
(ii) dissolving or dispersing phentermine or its pharmaceutically acceptable salt and one or more pharmaceutically acceptable excipients into a suitable solvent;
(iii) layering the extended-release topiramate cores with the solution or dispersion of phentermine; and
(iv) filling the cores prepared in step (iii) into a hard gelatin capsule or compressing the cores to form a tablet.

In one embodiment, the solvents used for preparing the solution or dispersion of phentermine include, but are not limited to, methylene chloride, isopropyl alcohol, acetone, methanol, ethanol, water, or mixtures thereof.

In another general aspect, the present invention relates to a method of treating obesity and related disorders by administering to a person in need thereof a pharmaceutical composition of phentermine and topiramate comprising:
(i) an extended-release portion comprising a therapeutically effective amount of topiramate or its pharmaceutically acceptable salt, one or more rate-controlling agents, and one or more pharmaceutically acceptable excipients;
(ii) an immediate-release portion comprising a therapeutically effective amount of phentermine or its pharmaceutically acceptable salt and one or more pharmaceutically acceptable excipients.

The details of one or more embodiments of the invention are set forth in the description below. Other features and objects of the invention will be apparent from the description and examples.

### DETAILED DESCRIPTION OF THE INVENTION

The object of the present invention is to provide a pharmaceutical composition comprising phentermine in an immediate-release (IR) form and topiramate in an extended-release (ER) form.

The present invention relates to a pharmaceutical composition of phentermine and topiramate comprising:
(i) an extended-release portion comprising a therapeutically effective amount of topiramate or its pharmaceutically acceptable salt, controlled-release polymers, and one or more pharmaceutically acceptable excipients;
(ii) an immediate-release portion comprising a therapeutically effective amount of phentermine or its pharmaceutically acceptable salt and one or more pharmaceutically acceptable excipients.

The term “therapeutically effective amount” of phentermine or topiramate refers to an amount in which the agent is nontoxic but sufficient to provide the desired effect.

The term “pharmaceutically acceptable salt” includes salts which are formed with inorganic acids, organic acids, or bases. Suitable salts include salts formed with hydrochloric, phosphoric, acetic, oxalic, tartaric, or mandelic acids, or those formed with bases such as sodium, potassium, ammonium, calcium, or ferric hydroxides.

The term “topiramate”, as used herein, would encompass topiramate base and all of its salts. Topiramate or its salt is present in the composition in an amount of from about 2% to about 85% by weight of the composition, more particularly from about 5% to about 75% by weight of the composition.

The term “phentermine”, as used herein, would encompass phentermine base and all of its salts. In particular, the pharmaceutical composition of the present invention comprises phentermine hydrochloride. Phentermine or its salt is present in the composition in an amount of from about 0.001% to about 40% by weight of the composition, more particularly, from about 0.1% to about 25% by weight of the composition.

The term “about”, as used herein, when used along with values assigned to certain measurements and parameters means a variation of up to 10% from such values, or in case of a range of values, means up to a 10% variation from both the lower and upper limits of such ranges.

The extended-release portion and the immediate-release portion may be present as cores which may be in the form of pellets, granules, beads, minitablets, or tablets. Alternatively, the extended-release topiramate cores may be coated with an immediate-release coating comprising a therapeutically effective amount of phentermine or its pharmaceutically acceptable salt. Further, the extended-release topiramate portion and the immediate-release phentermine portion may be encapsulated in a hard gelatin capsule. Alternatively, the extended-release topiramate portion and the immediate-release phentermine portion may be compressed into a tablet. The tablet may be a monolithic tablet, bilayered tablet, inlay tablet, or compression coated tablet.

The extended-release topiramate cores may be present as matrix cores. The matrix cores are prepared by blending topiramate or its pharmaceutically acceptable salt with one or more rate-controlling agents, and other pharmaceutically acceptable excipients, by processes known in the art, such as compaction, dry granulation, wet granulation, fluidized bed granulation, and extrusion-spheronization. The topiramate matrix cores may optionally be further coated with an extended-release polymer coating.

Alternatively, the extended-release topiramate cores may be present as reservoir cores produced by coating the immediate-release topiramate cores with an extended-release polymer coating.

The blend comprising topiramate or its pharmaceutically acceptable salt and one or more pharmaceutically acceptable excipients is layered onto inert cores as a powder, or as a solution or dispersion in a suitable solvent to form immediate-release topiramate cores which are then further coated with a layer of rate-controlling agents.

Alternatively, the immediate-release topiramate cores are prepared by extrusion-spheronization and are then further coated with a layer of rate-controlling agents.

Alternatively, the blend comprising topiramate or its pharmaceutically acceptable salt and one or more rate-controlling agents is layered onto inert cores as a powder or as a solution or dispersion in a suitable solvent by techniques known to one skilled in the art, such as drug layering, powder coating, roller compaction, granulation, or extrusion-spheronization.

The inert core may be water-soluble, water-swellable, water-insoluble, or mixtures thereof.

The water-soluble or water-swellable inert cores include one or more of sugar spheres or sugars like dextrose, lactose, anhydrous lactose, spray-dried lactose, lactose monohydrate, mannitol, starches, sorbitol, or sucrose. Tartaric acid pellets, or the commercially available inert core materials such as sugar sphere, non-pareil seed, and celphere may also be utilized.

Water-insoluble inert cores may include one or more of glass particles/beads, silicon dioxide, calcium phosphate dihydrate, dicalcium phosphate, calcium sulfate dihydrate, microcrystalline cellulose, cellulose derivatives, powdered cellulose, carnauba wax pellets, or mixtures thereof.

The extended-release core comprising topiramate or its pharmaceutically acceptable salt can be additionally coated with one or more non-rate controlling agents (seal layers) or rate-controlling agents (rate-controlling layers), or combinations of both. The location of the seal layers in the core is not critical. For example, the seal layers may be present over the inert core or may be present between the core and a rate-controlling layer. Alternatively, the seal layer may be coated over the rate-controlling layer.

The seal layers can be made of one or more polymers. Examples of polymers that can be used include, but are not limited to, hydroxypropyl methylcellulose, hydroxypropyl cellulose, methylcellulose, ethyl cellulose, polyvinyl alcohol, and polyethylene glycol. The seal layers may additionally contain other excipients such as plasticizers, pigments, or opacifiers. Examples of plasticizers that can be used include, but are not limited to, polyethylene glycols, glycerin, triacetin, triethyl citrate, diethyl phthalate, and mineral oils. Examples of pigments/opacifiers that can be used include, but are not limited to, water-soluble dyes, pigments, and natural products.

The rate-controlling agents used in the present invention can be hydrophilic, hydrophobic, or combinations of both.

Suitable examples of hydrophilic rate-controlling agents include, but are not limited to, cellulose derivatives such as hydroxypropyl cellulose, hydroxyethyl cellulose, hydroxymethyl cellulose, carboxymethylcellulose, methylcellulose, hydroxypropyl methylcellulose, sodium carboxy methylcellulose, or combinations thereof; polyvinylpyrrolidone; polyvinyl acetate, for example, Kollicoat® SR, copolymer of vinylpyrrolidone, and vinyl acetate; polysaccharides; polyalkylene glycols; starch; gums and derivatives; or mixtures thereof.

Suitable examples of hydrophobic rate-controlling agents include, but are not limited to, ethyl cellulose, ethyl cellulose aqueous dispersion, (e.g., Aquacoat® ECD-30 and Surelease®), cellulose acetate, cellulose acetate butyrate, poly(alkyl)methacrylate, hydroxypropyl methylcellulose phthalate, copolymers of acrylic or methacrylic acid esters, waxes, shellac, zein, castor oil, hydrogenated vegetable oils, or mixtures thereof.

The rate-controlling agents may additionally include plasticizers selected from polyethylene glycol, propylene glycol, triethylene glycol, ethyleneglycol monoleate, oleic acid, triethyl citrate, acetyl triethyl citrate, acetyl tributyl citrate, triacetin, diethyl phthalate, glyceryl monostearate, dibutyl sebacate, castor oil, or mixtures thereof.

The rate-controlling agents may further include pore-formers selected from polymers such as polyethylene glycol, polyvinyl pyrrolidone, polyvinyl alcohol, hydroxypropyl methyl cellulose, Kollicoat® IR (PVA/PEG copolymer 75:25 ratio), Eudragit® products, carrageenan, and alginates; salts such as sodium chloride, sodium phosphate, and calcium phosphate; and sugars such as sucrose, lactose, and mannitol.

The coating layers over the extended-release core may be applied as a solution or dispersion of coating ingredients using any conventional technique known in the art such as spray coating in a conventional coating pan or fluidized bed processor, or dip coating.

Example of solvents used for preparing a solution or dispersion of the coating ingredients include, but are not limited to, methylene chloride, isopropyl alcohol, acetone, methanol, ethanol, water, or mixtures thereof.

The immediate-release portion comprising phentermine may be present either in the form of a layer over the extended-release topiramate cores or may be present as separate units.

The layer of phentermine can be coated on the extended-release topiramate cores as a powder, or as a solution or dispersion in a suitable solvent, by any conventional technique known in the art such as pan coating, spray coating, centrifugal fluidized coating, or fluidized bed coating. Alternatively, the immediate-release coating may be applied by press-coating, dry compression, or deposition over the extended-release core or seal-coated extended-release core.

Example of solvents used for preparing a solution or dispersion of phentermine include, but are not limited to, methylene chloride, isopropyl alcohol, methanol, ethanol, acetone, water, or mixtures thereof.

Alternatively, the immediate-release portion comprising phentermine may be present as separate pellets, granules, beads, minitablets, or tablets, and may be prepared either by processes known in the art such as compaction, dry granulation, wet granulation, fluidized bed granulation, or extrusion-spheronization; or may be prepared by coating the phentermine solution or dispersion over the inert cores by techniques known to one skilled in the art, such as drug layering, powder coating, roller compaction, granulation, or extrusion-spheronization.

The extended-release and immediate-release pellets, granules, or beads are mixed with one or more pharmaceutically acceptable excipients and are either filled into a capsule or compressed into a tablet for ease of administration.

Both the extended-release core of topiramate and the immediate-release portion comprising phentermine may additionally contain suitable amounts of pharmaceutically acceptable excipients that would be necessary for preparing appropriate dosage forms. Examples of pharmaceutically acceptable excipients that can be used include, but are not limited to, diluents, binders, disintegrants, lubricants/glidants, buffers, wetting agents, wicking agents, coloring agents, and flavoring agents.

Suitable diluents may be selected from one or more of sugars, such as dextrose, glucose, sucrose, and lactose; sugar alcohols, such as sorbitol, xylitol, and mannitol; cellulose derivatives, such as powdered cellulose, microcrystalline cellulose, and silicified microcrystalline cellulose; starches, such as corn starch, pregelatinized starch, and maize starch; magnesium salts, such as oxides, carbonates, phosphates, calcium carbonate, calcium phosphate-dibasic, calcium phosphate-tribasic, and calcium sulfate; or mixtures thereof.

Suitable binders may be selected from one or more of starches, such as corn starch, pregelatinized starch, and maize starch; cellulose derivatives, such as hydroxypropyl methylcellulose, hydroxypropyl cellulose, hydroxyethyl cellulose, and methylcellulose; gums, such as xanthan gum, gum acacia, gum arabic, and tragacanth; water-soluble vinylpyrrolidone polymers, such as polyvinylpyrrolidone and copolymer of vinylpyrrolidone vinyl acetate; sugars, such as sorbitol and mannitol; sodium alginate; propylene glycol; methacrylates; or mixtures thereof.

Suitable disintegrants may be selected from one or more of alginic acid or alginates, microcrystalline cellulose, hydroxypropyl cellulose, croscarmellose sodium, crospovidone, sodium starch glycolate, starch, pregelatinized starch, carboxymethyl starch, polyacrylates, or mixtures thereof.

Suitable lubricants/glidants/anti-adherents may be selected from one or more of talc, Aerosil®, magnesium stearate, stearic acid, glyceryl monostearate, glyceryl behenate, sodium stearyl fumarate, sodium starch fumarate, or mixtures thereof.

Suitable buffers may be selected from one or more of phosphate, acetate, citrate, succinate, and histidine buffers.

Suitable wetting agents may be selected from one or more of hydrophilic or hydrophobic surfactants or mixtures thereof. The hydrophilic surfactants may be selected from one or more of non-ionic surfactants, ionic surfactants, or mixtures thereof.

Suitable wicking agents may be selected from one or more of silicon dioxide (such as Syloid® 244 FP), kaolin, titanium dioxide, alumina, niacinamide, sodium lauryl sulfate, low molecular weight polyvinylpyrrolidone, m-pyrol, bentonite, magnesium aluminum silicate, polyester, polyethylene, or mixtures thereof.

Suitable coloring and flavoring agents may be selected from any FDA approved colors for oral use.

Suitable solvents used in the preparation of the composition of the present invention include, but are not limited to, methylene chloride, isopropyl alcohol, acetone, methanol, ethanol, water, or mixtures thereof.

The composition of the present invention may optionally be coated with one or more layers comprising film-forming agents and/or pharmaceutically acceptable excipients.

The pharmaceutical composition of the present invention may be used for treating obesity and related disorders. The pharmaceutical composition of the present invention may contain an additional therapeutic agent, or may be administered in combination with other therapeutic agents.

The present invention is illustrated below by reference to the following example. However, one skilled in the art will appreciate that the specific methods and results discussed are merely illustrative of the invention, and are not to be construed as limiting the invention.

### EXAMPLE 1

| | |
|---|---|
| | Percent w/w of |
| Ingredients | Formulation |

Topiramate Pellets

| | |
|---|---|
| Topiramate | 44.9 |
| Microcrystalline cellulose | 24.4 |
| Hydroxypropyl methylcellulose | 3.9 |
| Purified water | q.s. |

Extended-Release (ER) Coating

| | |
|---|---|
| Ethyl cellulose | 5.3 |
| Polyethylene glycol | 1.0 |
| Hydroxypropyl methylcellulose | 1.0 |
| Isopropyl alcohol + purified water | q.s. |

Phentermine Immediate-Release (IR) Coating

| | |
|---|---|
| Phentermine | 7.3 |
| Lactose | 4.8 |
| Polyvinylpyrrolidone | 4.9 |
| Isopropyl alcohol/purified water or combination of both | q.s. |

Film Coating (optional)

| | |
|---|---|
| Opadry ® coating | 2.0 |
| Purified water | q.s. |

Lubrication

| | |
|---|---|
| Magnesium stearate | 0.5 |

### Procedure:

### Preparation of Topiramate Extended-Release Spheroids

1. Topiramate and microcrystalline cellulose were sifted through a suitable screen and the mixture was blended for 15 minutes.
2. A binder solution was prepared by dissolving hydroxypropyl methylcellulose in water.
3. The binder solution of step 2 was added to the blend of step 1.
4. The wet mass of step 3 was transferred into the extruder and extrudes were prepared and then transferred into the spheroniser and spheronised.
5. The spheroids of step 4 were dried at about 60° C. until the loss on drying (at 105° C. for 10 minutes) was between 1.0% to 3.0% w/w, and were then sifted through suitable screens to remove oversize spheroids and fines.

### Extended-Release (ER) Coating

6. Ethyl cellulose was dispersed in isopropyl alcohol and purified water followed by polyethylene glycol and hydroxypropyl methylcellulose under continuous stirring.
7. The spheroids of step 5 were coated with the solution of step 6 until the target weight build-up.
8. The spheroids of step 7 were dried until the loss on drying (at 105° C. for 10 minutes) was between 1.0% to 3.0% w/w, and were then sifted through suitable screens to remove oversize spheroids and fines.

### Phentermine Immediate-Release (IR) Coating

9. To isopropyl alcohol/purified water, or a combination of both, polyvinylpyrrolidone was slowly added under stirring to ensure no lumps were formed.
10. Phentermine hydrochloride was added slowly to the solution of step 9 to ensure no lumps were formed, and stirring was continued for another 30 minutes. Lactose was added to the same dispersion and stirred for 15 to 30 minutes to dissolve the lactose.
11. The topiramate extended-release spheroids of step 8 were loaded in the Wurster and layered with the phentermine solution of step 10 until target weight gain.
12. The pellets of step 11 were dried and sifted through suitable screens.

### Opadry® Film-Coating (Optional)

13. 12% w/w aqueous dispersion of Opadry® was prepared.
14. The phentermine-layered pellets of step 12 were loaded into the Wurster and film-coating with the Opadry® dispersion was continued until the target weight gain was attained.

### Lubrication

15. Magnesium stearate was sifted and the coated pellets of step 14 were lubricated.

### Capsule Filling

16. The lubricated pellets of step 15 were filled into hard gelatin capsules using suitable capsule filling equipment.

### EXAMPLE 2

| | |
|---|---|
| | Percent w/w of |
| Ingredients | Formulation |

Topiramate Pellets

| | |
|---|---|
| Topiramate | 40.0 |
| Microcrystalline cellulose | 21.5 |
| Hydroxypropyl methylcellulose | 3.3 |
| Purified water | q.s. |

ER Coating

| | |
|---|---|
| Ethyl cellulose | 4.5 |
| Polyethylene glycol | 0.9 |
| Hydroxypropyl methylcellulose | 0.9 |
| Isopropyl alcohol + purified water | q.s. |

Phentermine IR Pellets

| | |
|---|---|
| Inert core (Seal coated with hydroxypropyl | 14.5 |
| methylcellulose E5, optionally); Sugar spheres | |

Drug Loading

| | |
|---|---|
| Phentermine | 6.2 |
| Lactose | 4.1 |
| Polyvinylpyrrolidone | 4.1 |
| Isopropyl alcohol/purified water or combination of both | q.s. |

### Procedure:

### Preparation of Topiramate Extended-Release Spheroids

1. Topiramate extended-release spheroids were prepared in a similar way as described in Example 1.

### Preparation of Phentermine Immediate-Release Pellets

2. To isopropyl alcohol/purified water or combination of both, polyvinylpyrrolidone was slowly added under stirring to ensure no lumps were formed.
3. Phentermine hydrochloride was added slowly to step 2 to ensure no lumps were formed and stirring was continued for a further 30 minutes. Lactose was added to the same dispersion and stirred for 15 to 30 minutes to dissolve the lactose.
4. Sugar spheres (optionally seal coated with hydroxypropyl methylcellulose) were loaded into the Wurster, and the drug layering process was continued until the target weight gain was attained.
5. The spheroids of step 4 were dried and sifted through suitable screens.

### Capsule Filling

6. The extended-release pellets of topiramate and the immediate-release pellets of phentermine were filled into hard gelatin capsules using suitable capsule filling equipment.

### EXAMPLE 3

| | |
|---|---|
| | Percent w/w of |
| Ingredients | Formulation |

Topiramate Pellets

| | |
|---|---|
| Topiramate | 46.0 |
| Microcrystalline cellulose | 25.0 |
| Hydroxypropyl methylcellulose | 4.0 |
| Purified water | q.s. |

ER Coating

| | |
|---|---|
| Ethyl cellulose | 5.4 |
| Polyethylene glycol | 1.1 |
| Hydroxypropyl methylcellulose | 1.1 |
| Isopropyl alcohol + purified water | q.s. |

Phentermine IR Pellets/Granules

| | |
|---|---|
| Phentermine | 7.5 |
| Microcystalline cellulose | 6.0 |
| Croscarmellose sodium | 0.7 |
| Hydroxypropyl methylcellulose | 0.7 |
| Purified water | q.s. |

Film-Coating (optional)

| | |
|---|---|
| Opadry ® coating | 2.0 |
| Purified water | q.s. |

Lubrication

| | |
|---|---|
| Magnesium stearate | 0.5 |

### Procedure:

### Preparation of Topiramate Extended-Release Spheroids

1. Topiramate extended-release spheroids were prepared in a similar way as described in Example 1.

### Preparation of Phentermine Immediate-Release Granules

2. Phentermine hydrochloride and microcrystalline cellulose were weighed and blended.
3. The blend of step 2 was granulated using hydroxypropyl methylcellulose in purified water as the binder solution.
4. The granules were dried to a loss on drying of not more than 4% to 6% and blended with croscarmellose sodium.
5. The blend of step 4 was lubricated using magnesium stearate.

### Opadry® Film-Coating (Optional)

6. A 12% w/w aqueous dispersion of Opadry® was prepared.
7. The phentermine granules of step 4 were loaded into the Wurster and the film-coating with Opadry® dispersion was continued until target weight gain.

### Lubrication

8. Magnesium stearate was sifted, and the coated granules of step 7 were lubricated.

### Capsule Filling

9. The extended-release pellets of topiramate and the immediate-release granules of phentermine were filled into hard gelatin capsules using suitable capsule filling equipment.

### EXAMPLE 4

| | |
|---|---|
| | Percent w/w of |
| Ingredients | Formulation |

Topiramate Pellets

| | |
|---|---|
| Inert core (seal-coated with hydroxypropyl | 38.8 |
| methylcellulose E5, optionally); sugar spheres | |

Drug Loading

| | |
|---|---|
| Topiramate | 32.4 |
| Mannitol (optional) | |
| Hydroxypropyl methylcellulose | 6.3 |
| Isopropyl alcohol/purified water or combination of both | q.s. |

ER Coating

| | |
|---|---|
| Ethyl cellulose | 5.6 |
| Polyethylene glycol | 1.1 |
| Hydroxypropyl methylcellulose | 1.1 |
| Isopropyl alcohol + purified water | q.s. |

Phentermine IR Coating

| | |
|---|---|
| Phentermine | 5.3 |
| Lactose | 3.5 |
| Polyvinylpyrrolidone | 3.5 |
| Isopropyl alcohol/purified water or combination of both | q.s. |

Film-Coating (optional)

| | |
|---|---|
| Opadry ® coating | 1.9 |
| Purified water | q.s. |

Lubrication

| | |
|---|---|
| Magnesium stearate | 0.5 |

### Procedure:

### Preparation of Topiramate Extended-Release Pellets

1. A seal-coating solution of hydroxypropyl methylcellulose in purified water was prepared. Sugar spheres were loaded into the Wurster and seal coated until the target weight gain was attained.
2. The pellets of step 1 were dried and sifted through suitable screens to remove oversize pellets and fines.
3. To isopropyl alcohol, hydroxypropyl methylcellulose was added slowly under stirring to ensure no lumps were formed, and stirring was continued for 10 minutes.
4. Topiramate was added to the solution of step 3 slowly to ensure no lumps were formed, and stirring was continued for further 30 minutes. Lactose was added to the same dispersion, and stirred for 15 to 30 minutes to dissolve the lactose. The dispersion was passed through a suitable screen.
5. The seal-coated sugar spheres of step 2 were loaded into the Wurster, and the drug layering process was continued until target weight gain.
6. The drug loaded pellets of step 5 were dried at about 60° C. until the loss on drying (at 105° C. for 10 minutes) was between 1.0% to 3.0% w/w, and sifted through suitable screens to remove oversize spheroids and fines.

### Extended-Release Coating

7. Ethyl cellulose was dispersed in isopropyl alcohol and purified water followed by polyethylene glycol and hydroxypropyl methylcellulose under continuous stirring.
8. The pellets of step 6 were coated with the solution of step 7 until the target weight build up.
9. The pellets of step 8 were dried until the loss on drying (at 105° C. for 10 minutes) was between 1.0% to 3.0% w/w and were then sifted through suitable screens to remove oversize spheroids and fines.

### Phentermine IR Coating

10. The topiramate extended-release pellets were drug layered with phentermine solution in a similar way as described in Example 1.

### Capsule Filling

11. The lubricated pellets were filled into hard gelatin capsules using suitable capsule filling equipment.

### EXAMPLE 5

| | |
|---|---|
| | Percent w/w of |
| Ingredients | Formulation |

Topiramate Pellets

| | |
|---|---|
| Inert core (seal-coated with hydroxypropyl | 38.0 |
| methylcellulose E5, optionally); sugar spheres | |

Drug Loading

| | |
|---|---|
| Topiramate | 28.0 |
| Lactose (filler optional) | |
| Hydroxypropyl methylcellulose | 5.4 |
| Isopropyl alcohol/purified water or combination of both | q.s. |

ER Coating

| | |
|---|---|
| Ethyl cellulose | 4.6 |
| Polyethylene glycol | 1.0 |
| Hydroxypropyl methylcellulose | 1.0 |
| Isopropyl alcohol + purified water | q.s. |

Phentermine IR Pellets

| | |
|---|---|
| Inert core (seal-coated with hydroxypropyl | 9.2 |
| methylcellulose E5, optionally); sugar spheres | |

Drug Loading

| | |
|---|---|
| Phentermine | 5.2 |
| Lactose | 3.8 |
| Polyvinylpyrrolidone | 3.8 |
| Isopropyl alcohol/purified water or combination of both | q.s. |

### Procedure:

### Preparation of Topiramate Extended-Release Pellets

1. Topiramate extended-release pellets were prepared in a similar way as described in Example 4.

### Preparation of Phentermine Immediate-Release Pellets

2. Phentermine immediate-release pellets were prepared in a similar way as described in Example 2.

### Capsule Filling

3. The extended-release pellets of topiramate and the immediate-release pellets of phentermine were filled into hard gelatin capsules using suitable capsule filling equipment.

### EXAMPLE 6

| | |
|---|---|
| | Percent w/w of |
| Ingredients | Formulation |

Topiramate Pellets

| | |
|---|---|
| Inert core (seal-coated with hydroxypropyl | 38.0 |
| methylcellulose E5, optionally); sugar spheres | |

Drug Loading

| | |
|---|---|
| Topiramate | 31.4 |
| Lactose (optional) | |
| Hydroxypropyl methylcellulose | 5.8 |
| Isopropyl alcohol/purified water or combination of both | q.s. |

ER Coating

| | |
|---|---|
| Ethyl cellulose | 5.1 |
| Polyethylene glycol | 1.1 |
| Hydroxypropyl methylcellulose | 1.1 |
| Isopropyl alcohol + purified water | q.s. |

Phentermine IR Pellets/Granules

| | |
|---|---|
| Phentermine | 7.5 |
| Microcrystalline cellulose | 6.0 |
| Croscarmellose sodium | 0.7 |
| Hydroxypropyl methylcellulose | 0.8 |
| Purified water | q.s. |

Film Coating (optional)

| | |
|---|---|
| Opadry ® coating | 2.0 |
| Purified water | q.s. |

Lubrication

| | |
|---|---|
| Magnesium stearate | 0.5 |

### Procedure:

### Preparation of Topiramate Extended-Release Pellets

1. Topiramate extended-release pellets were prepared in a similar way as described in Example 4.

### Preparation of Phentermine Immediate-Release Granules

2. Phentermine immediate-release granules were prepared in a similar way as described in Example 3.

### Capsule Filling

3. The extended-release pellets of topiramate and the immediate-release granules of phentermine were filled into hard gelatin capsules using suitable capsule filling equipment.

### EXAMPLE 7

| | |
|---|---|
| | Percent w/w of |
| Ingredients | Formulation |

Topiramate-Polymer Matrix Coating Pellets

| | |
|---|---|
| Inert core (seal-coated with hydroxypropyl | 39.7 |
| methylcellulose E5, optionally); sugar spheres | |

Drug Polymer Matrix Loading

| | |
|---|---|
| Topiramate | 26.8 |
| Lactose | |
| Polyvinylpyrrolidone (optional) | 6.6 |
| Ethyl cellulose | 5.3 |
| Polyethylene glycol | 1.1 |
| Isopropyl alcohol + purified water | q.s. |

ER Coating (optional)

| | |
|---|---|
| Ethyl cellulose | 5.7 |
| Polyethylene glycol | 1.1 |
| Hydroxypropyl methylcellulose | 1.1 |
| Isopropyl alcohol + purified water | q.s. |

Phentermine IR Coating

| | |
|---|---|
| Phentermine | 4.3 |
| Lactose | 2.9 |
| Croscarmellose sodium | 0.9 |
| Polyvinylpyrrolidone | 2.9 |
| Isopropyl alcohol/purified water or combination of both | q.s. |

Film Coating (optional)

| | |
|---|---|
| Opadry ® coating | 2.0 |
| Purified water | q.s. |

Lubrication

| | |
|---|---|
| Magnesium stearate | 0.5 |

### Procedure:

### Preparation of Topiramate-Polymer Matrix Coating Pellets

### Preparation of Topiramate Drug-Layered Pellets

1. A seal-coating solution of hydroxypropyl methylcellulose in purified water was prepared. Sugar spheres were loaded into the Wurster and seal coated until target weight gain.
2. The pellets of step 1 were dried and sifted through suitable screens to remove oversize and fines.
3. To isopropyl alcohol, ethyl cellulose and polyvinylpyrrolidone were added slowly under stirring to ensure no lumps were formed, and stirring was continued for 10 minutes.
4. Topiramate was added to the solution of step 3 slowly to ensure no lumps were formed and stirring was continued for further 30 minutes. Lactose was added to the same dispersion and stirred for 15 to 30 minutes to dissolve the lactose. The dispersion was passed through suitable screens.
5. The seal-coated sugar spheres of step 2 were loaded into the Wurster and the drug layering process was continued until target weight gain.
6. The drug loaded pellets of step 5 were dried at about 60° C. until the loss on drying (at 105° C. for 10 minutes) was between 1.0% to 3.0% w/w and sifted through suitable screens to remove oversize and fines.

### Extended-Release Coating (Optional)

7. Ethyl cellulose was dispersed in isopropyl alcohol and purified water followed by polyethylene glycol and hydroxypropyl methylcellulose under continuous stirring.
8. The pellets of step 6 were coated with the solution of step 7 until the target weight build up was attained.
9. The pellets of step 8 were dried until the loss on drying (at 105° C. for 10 minutes) was between 1.0% to 3.0% w/w and then sifted through suitable screens to remove oversize and fines.

### Phentermine IR Coating

10. The topiramate-polymer matrix coating pellets were drug-layered with phentermine solution in a similar way as described in Example 1.

### Capsule Filling

11. The lubricated pellets were filled into hard gelatin capsules using suitable capsule filling equipment.

### EXAMPLE 8

| | |
|---|---|
| | Percent w/w of |
| Ingredients | Formulation |

Topiramate-Polymer Matrix Coating Pellets

| | |
|---|---|
| Topiramate | 39.8 |
| Microcrystalline cellulose | 21.9 |
| Polyvinylpyrrolidone | 3.5 |
| Ethyl cellulose | 8.5 |
| Polyethylene glycol | 1.6 |
| Purified water | q.s. |

ER Coating (optional)

| | |
|---|---|
| Ethyl cellulose | 5.6 |
| Polyethylene glycol | 1.1 |
| Hydroxypropyl methylcellulose | 1.1 |
| Isopropyl alcohol + purified water | q.s. |

Phentermine IR Coating

| | |
|---|---|
| Phentermine | 6.5 |
| Lactose | 3.5 |
| Polyvinylpyrrolidone | 4.5 |
| Isopropyl alcohol/purified water or combination of both | q.s. |

Film Coating (optional)

| | |
|---|---|
| Opadry ® coating | 1.9 |
| Purified water | q.s. |

Lubrication

| | |
|---|---|
| Magnesium stearate | 0.5 |

### Procedure:

### Preparation of Topiramate-Polymer Matrix Coating Pellets

### Preparation of Topiramate Drug-Layered Pellets

1. Topiramate, microcrystalline cellulose, polyethylene glycol, and ethyl cellulose were sifted through suitable screens.
2. A binder solution was prepared by dissolving polyvinylpyrrolidone in purified water.
3. The binder solution of step 2 was added to the blend of step 1 in a rapid mixer granulator over a period of 3 to 5 minutes.
4. The wet mass of step 3 was transferred into the extruder and extrudes were prepared and then transferred into the spheroniser and spheronised.
5. The spheroids of step 4 were dried at about 60° C. until the loss on drying (at 105° C. for 10 minutes) was between 1.0% to 3.0% w/w and then sifted through suitable screens to remove oversize spheroids and fines.

### Extended-Release Coating (Optional)

6. Ethyl cellulose was dispersed in isopropyl alcohol and purified water followed by polyethylene glycol and hydroxypropyl methylcellulose under continuous stirring.
7. The pellets of step 5 were coated with the solution of step 6 until the target weight build up was attained.
8. The pellets of step 7 were dried until the loss on drying (at 105° C. for 10 minutes) was between 1.0% to 3.0% w/w and then sifted through suitable screens to remove oversize and fines.

### Phentermine IR Coating

9. The topiramate-polymer matrix coating pellets were drug-layered with phentermine solution by a process as described in Example 1.

### Capsule Filling

10. The lubricated pellets were filled into hard gelatin capsules using suitable capsule filling equipment.

### EXAMPLE 9

| | |
|---|---|
| | Percent w/w of |
| Ingredients | Formulation |

Topiramate Pellets

| | |
|---|---|
| Topiramate | 18.15 |
| Microcrystalline cellulose | 25.85 |
| Polyvinylpyrrolidone | 1.38 |
| Purified water | q.s. |

Seal Coating

| | |
|---|---|
| Hydroxypropyl methylcellulose | 1.36 |
| Purified water | q.s. |

ER Coating

| | |
|---|---|
| Ethyl cellulose | 2.19 |
| Polyvinylpyrrolidone | 1.46 |
| Dibutyl sebacate | 0.55 |
| Isopropyl alcohol + purified water | q.s. |

Lubrication

| | |
|---|---|
| Talc | 0.53 |

Phentermine IR Pellets

| | |
|---|---|
| Sugar beads | 43.41 |

Drug Loading

| | |
|---|---|
| Phentermine | 2.56 |
| Hydroxypropyl methylcellulose | 2.56 |
| Purified water | q.s. |

### Procedure:

### Preparation of Topiramate Extended-Release Spheroids

1. Topiramate and microcrystalline cellulose were sifted through a suitable screen and the mixture was blended.
2. A binder solution was prepared by dispersing polyvinylpyrrolidone in water under stirring.
3. The binder solution of step 2 was added to the blend of step 1.
4. The wet mass of step 3 was transferred into the extruder and extrudes were prepared and then transferred into the spheroniser and spheronised.
5. The spheroids of step 4 were dried at about 60° C. until the loss on drying (at 105° C. for 10 minutes) was not more than 2% w/w and then sifted through suitable screens to remove oversize spheroids and fines.

### Seal Coating

6. An aqueous binder solution was prepared by dispersing hydroxypropyl methylcellulose in purified water under stirring.
7. The binder solution was sprayed over the spheroids of step 5 to a weight gain of 2% to 4% and the seal coated spheroids were dried.

### Extended-Release Coating

8. Polyvinylpyrrolidone was dispersed in isopropyl alcohol and purified water followed by the addition of ethyl cellulose and dibutyl sebacate under continuous stirring to get a clear solution.
9. The spheroids of step 7 were coated with the solution of step 8 until the target weight build up was attained.
10. The spheroids of step 9 were dried at about 50° C. to 60° C. until the loss on drying (at 105° C. for 10 minutes) was not more than 2% w/w.

### Lubrication

11. Talc was sifted and the coated pellets of step 10 were lubricated.

### Preparation of Phentermine Immediate-Release Pellets

12. Hydroxypropyl methylcellulose was dissolved in water followed by the addition of phentermine hydrochloride under continuous stirring.
13. Sugar spheres were loaded into the Wurster and the drug layering process was continued until target weight gain.
14. The spheroids of step 13 were dried and sifted through suitable screens.

### Capsule Filling

15. The extended-release pellets of topiramate and the immediate-release pellets of phentermine were filled into hard gelatin capsules using suitable capsule filling equipment.

### Dissolution Studies:

Table 1 provides the dissolution data of topiramate capsules of 92 mg strength prepared as per Example 9. The dissolution was carried out using USP Type I apparatus at 100 rpm at a temperature of 37° C.±0.5° C. wherein 500 mL of acetate buffer of pH 4.5 was used as the dissolution medium.

TABLE 1

| | Time (hours) | | | | | | |
|---|---|---|---|---|---|---|---|
| | 1 | 2 | 4 | 6 | 8 | 10 | 12 |
| Percent Drug-Release | 11 | 20 | 35 | 51 | 59 | 67 | 78 |

### EXAMPLE 10

| | |
|---|---|
| | Percent w/w of |
| Ingredients | Formulation |

Topiramate Pellets

| | |
|---|---|
| Topiramate | 17.07 |
| Microcrystalline cellulose | 24.31 |
| Polyvinylpyrrolidone | 1.30 |
| Purified water | q.s. |

Seal Coating

| | |
|---|---|
| Hydroxypropyl methylcellulose | 1.28 |
| Purified water | q.s. |

ER Coating

| | |
|---|---|
| Ethyl cellulose aqueous dispersion | 8.51 |
| Purified water | q.s. |
| Polyvinylpyrrolidone | 0.64 |
| Dibutyl sebacate | 0.77 |

Lubrication

| | |
|---|---|
| Talc | 0.48 |

Phentermine IR Pellets

| | |
|---|---|
| Sugar beads | 40.82 |

Drug Loading

| | |
|---|---|
| Phentermine | 2.41 |
| Hydroxypropyl methylcellulose | 2.41 |
| Purified water | q.s. |

### Procedure:

### Preparation of Topiramate Extended-Release Spheroids

1. Topiramate extended-release spheroids were prepared by a process as described in Example 9 except for the extended-release coating which comprise the following steps:
2. Ethyl cellulose aqueous dispersion and dibutyl sebacate were mixed in a container under continuous stirring.
3. Polyvinylpyrrolidone was dissolved in water and the aqueous polyvinylpyrrolidone solution was slowly added to the ethyl cellulose dispersion of step 2 under continuous stirring.

### Preparation of Phentermine Immediate-Release Pellets

4. Phentermine immediate-release pellets were prepared by a process as described in Example 9.

### Capsule Filling

5. The extended-release pellets of topiramate and the immediate-release pellets of phentermine were filled into hard gelatin capsules using suitable capsule filling equipment.

### Dissolution Studies:

Table 2 provides the dissolution data of Topiramate capsules of 92 mg strength prepared as per Example 10. The dissolution was carried out using USP Type I apparatus at 100 rpm at a temperature of 37° C.±0.5° C. wherein 500 mL of acetate buffer of pH 4.5 was used as the dissolution medium.

TABLE 2

| | Time (hours) | | | | | | |
|---|---|---|---|---|---|---|---|
| | 1 | 2 | 4 | 6 | 8 | 10 | 12 |
| Percent Drug-Release | 14 | 29 | 53 | 70 | 82 | 90 | 95 |

While several particular forms of the invention have been illustrated and described, it will be apparent that various modifications and combinations of the invention detailed in the text can be made without departing from the spirit and scope of the invention.

We claim:

1. A pharmaceutical composition of phentermine and topiramate comprising:
(i) an extended-release portion comprising a therapeutically effective amount of topiramate or its pharmaceutically acceptable salt, one or more rate-controlling agents, and one or more pharmaceutically acceptable excipients;
(ii) an immediate-release portion comprising a therapeutically effective amount of phentermine or its pharmaceutically acceptable salt and one or more pharmaceutically acceptable excipients.

2. The pharmaceutical composition according to claim 1, wherein the extended-release portion and the immediate-release portion are present as cores which may be in the form of pellets, granules, beads, minitablets, or tablets.

3. The pharmaceutical composition according to claim 1, wherein the extended-release topiramate portion is present as cores and the immediate-release phentermine portion is present as a coating over the extended-release portion.

4. The pharmaceutical composition according to claim 1, wherein the extended-release topiramate cores are present as matrix cores with an optional extended-release polymer coating over the cores.

5. The pharmaceutical composition according to claim 1, wherein the extended-release topiramate cores are present as reservoir cores produced by coating the immediate-release topiramate cores with an extended-release polymer coating.

6. The pharmaceutical composition according to claim 1, wherein the pharmaceutical composition of the present invention is a hard gelatin capsule or a tablet.

7. A process of preparing a pharmaceutical composition of phentermine and topiramate comprising the following steps:
(i) preparing extended-release topiramate cores comprising a therapeutically effective amount of topiramate or its pharmaceutically acceptable salt, one or more rate-controlling agents, and one or more pharmaceutically acceptable excipients;
(ii) preparing immediate-release phentermine cores comprising a therapeutically effective amount of phentermine or its pharmaceutically acceptable salt and one or more pharmaceutically acceptable excipients; and
(iii) combining the extended-release topiramate cores and the immediate-release phentermine cores.

8. The process according to claim 7, wherein the extended-release topiramate cores are prepared by compaction, dry granulation, wet granulation, fluidized bed granulation, or extrusion-spheronization.

9. A process of preparing a pharmaceutical composition of phentermine and topiramate, wherein the process comprises the steps of:
(i) preparing extended-release topiramate cores comprising a therapeutically effective amount of topiramate or its pharmaceutically acceptable salt, one or more rate-controlling agents, and one or more pharmaceutically acceptable excipients;
(ii) dissolving or dispersing phentermine or its pharmaceutically acceptable salt and one or more pharmaceutically acceptable excipients into a suitable solvent;
(iii) layering the extended-release topiramate cores with the solution or dispersion of phentermine; and
(iv) filling the cores prepared in step (iii) into a hard gelatin capsule or compressing the cores to form a tablet.

10. A method of treating obesity and related disorders by administering to a person in need thereof a pharmaceutical composition of phentermine and topiramate comprising:
(i) an extended-release portion comprising a therapeutically effective amount of topiramate, one or more rate-controlling agents, and one or more pharmaceutically acceptable excipients;
(ii) an immediate-release portion comprising a therapeutically effective amount of phentermine and one or more pharmaceutically acceptable excipients.
